(19) United States
(12) Patent Application Publication
(10) Pub. No.: US 2024/0168013 A1
(43) Pub. Date: May 23, 2024

(54) Cellular Immunity Assay by Topical Application of Pathogen Particles

(71) Applicant: VaxDome Inc., Fort Worth, TX (US)

(72) Inventor: De-chu Christopher Tang, Fort Worth, TX (US)

(21) Appl. No.: 18/513,946

(22) Filed: Nov. 20, 2023

Related U.S. Application Data
(60) Provisional application No. 63/427,231, filed on Nov. 22, 2022.

(51) Int. Cl. G01N 33/50

(52) U.S. Cl. CPC G01N 33/5091; G01N 33/5088

(57) ABSTRACT

Provided herein are methods of measuring a cellular immune response comprising: topically applying pathogen particles or tumor cells to a site on an outermost layer of skin, wherein the pathogen particles or tumor cells can recruit immune cells to the administration site; imaging the changes in cutaneous architecture driven by infiltration of immune cells at the site of administration after at least one day from the applying step; and characterizing one or more structural changes to one or more cutaneous layers at the site administration using an imager.

### CROSS-REFERENCE TO RELATED APPLICATIONS

This application claims priority to U.S. Provisional Application Ser. No. 63/427,231, filed Nov. 22, 2022, the entire contents of which are incorporated herein by reference.

### TECHNICAL FIELD OF THE INVENTION

The present invention relates in general to the field of immunity assay, and more particularly, to a novel composition and method for detecting cellular immune responses to specific pathogens and tumors.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with immunity assays.

In addition to antibodies, compelling evidence shows that the functionality of the T-cell response has an important bearing on the degree of protection in many disease settings. However, the contemporary cellular immunity assays including stimulation of peripheral blood mononuclear cells (PBMC) from vaccinated subjects with whole pathogens/lysates/infected cells and antigen-specific assays such as enzyme-linked immunosorbent spot (ELISPOT) assay, peptide pools, tetramers, etc., are cumbersome to perform (Davis, 2020; Flaxman and Ewer, 2018). Further, these assays demonstrate an undesirable bias associated with the missing-in-action (MIA) nonfighter T cells commonly found in PBMC (Milner et al., 2017; Shin and Iwasaki, 2013). The failure to distinguish tissue-resident fighter T cells from their MIA nonfighter counterparts that tend to circulate (Milner et al., 2017) when both classes of T cells bind the same epitopes causes the inconsistencies associated with contemporary cellular immunity assays. Likewise, antibody-based humoral immunity assays are also not comprehensive as they are not well correlated with protective immunity (Takahashi and Iwasaki, 2021; Theel et al., 2020; Wang et al., 2020; Wright et al., 2016).

For example, although serum antibody titer has been accepted as a correlate for protective immunity elicited by trivalent inactivated influenza vaccine (TIV), vaccination by nasal spray of live attenuated influenza vaccine (LAIV; e.g., FluMist) elicits potent protective immunity that is not correlated with serum antibody titers or any known surrogate markers (Wright et al., 2016). In addition, no surrogate immunity assay that measures the degree of resistance to severe acute respiratory syndrome coronavirus 2 (SARS-CoV-2) infection has been developed (Gilbert et al., 2022).

As such, a need remains for novel immunity assays that can be used to gauge the potency of a cellular immune response by recruiting tissue-resident immune cells to specific pathogen particles underneath a patch within a subset of skin.

### SUMMARY OF THE INVENTION

As embodied and broadly described herein, an aspect of the present disclosure relates to a method of measuring a cellular immune response, the method comprising: topically applying pathogen particles or tumor cells to a site on an outermost layer of skin, wherein the pathogen particles or tumor cells can recruit immune cells to a site of administration; imaging changes in cutaneous architecture driven by infiltration of immune cells at the site after at least one day from the applying step; and characterizing one or more structural changes to one or more cutaneous layers at the site of administration using an imager. In one aspect, the pathogen particles comprise pathogens inactivated by γ-irradiation. In another aspect, the pathogen particles comprise pathogens inactivated by H₂O₂. In another aspect, the pathogen particles comprise pathogens inactivated by heat. In another aspect, the pathogen particles comprise pathogens selected from a virus, a bacterium, or a fungus. In another aspect, the virus is selected from: influenza virus, coronavirus, respiratory syncytial virus, rhinovirus, or measles virus. In another aspect, the bacterium is selected from: Bacillus, Clostridium, Mycobacterium, Staphylococcus, Streptococcus, Pseudomonas, Klebsiella, Haemophilus, or Mycoplasma. In another aspect, the fungus is selected from: Aspergillus flavus, Aspergillus fumigatus, Aspergillus nidulans, Aspergillus niger, Aspergillus terreus, Aspergillus ustus, Candida albicans, Candida alibicans, Candida glabrata, Candida lipolytica, Candida tropicalis, Candida tropicalis, Cryptococcus neoformans, Cryptococcus neoformas, Fusarium moniliforme, Geotricum candidum, Microsporum canis, Mucor circillelloides, Penicillium aurantiogriseum, Penicillium expansum, Penicillium italicum, Penicillium marneffei, Penicilium marneffeii, Rhizopus oryzaee, Sporotlirix schenckii, Syncephalastrum racemosum, Trichophyton mentagrophytes, Trichophyton rubrum, and a combination thereof. In another aspect, the tumor cells are derived from a tumor biopsy resected from a patient or cultured tumor cells. In another aspect, the method further comprises staining the one or more cutaneous layers at the site of administration prior to the imaging step. In another aspect, the method further comprises determining a number and type of recruited immune cells and immune cell-induced structural changes in one or more cutaneous layers at the site of administration, wherein the immune cells are impartial markers for determining a potency of a pathogen/tumor-specific cellular immune response without bias associated with nonfighter T cells. In another aspect, the method further comprises detecting a protective cellular immune response against a wide variety of pathogens without contamination by nonfighter T cells, wherein an assay is selected from at least one of a quantitative, a noninvasive, or a personalized assay.

As embodied and broadly described herein, an aspect of the present disclosure relates to a composition for detecting a protective cellular immune response comprising: live or inactivated virus particles, bacterium cells, fungus cells, or tumor cells that recruit immune cells to a site of administration, wherein inactivation is by heat, γ-irradiation, or H₂O₂ formulated for topical administration at a site of administration for detection of the protective cellular immune response. In one aspect, the composition further comprises an abrasive.

As embodied and broadly described herein, an aspect of the present disclosure relates to a method of measuring a cellular immune response, the method comprising: topically applying pathogen particles or tumor cells to a site of administration on an outermost layer of skin, wherein the pathogen particles or tumor cells can recruit immune cells to the site of administration; imaging changes in cutaneous architecture driven by infiltration of immune cells at the site after at least one day from the applying step; characterizing one or more structural changes to one or more cutaneous layers at the site administration using an imager; and detecting a protective cellular immune response against the pathogen particles or tumor cells, wherein the assay is selected from at least one of a quantitative, a noninvasive, or a personalized assay. In one aspect, the pathogen particles comprise pathogens inactivated by γ-irradiation. In another aspect, the pathogen particles comprise pathogens inactivated by H₂O₂. In another aspect, the pathogen particles comprise pathogens inactivated by heat. In another aspect, the pathogen is a virus, a bacterium, a fungus, or a tumor cell. In another aspect, the pathogen is a virus selected from: influenza virus, coronavirus, respiratory syncytial virus, rhinovirus, or measles virus. In another aspect, the pathogen is a bacterium selected from: Bacillus, Clostridium, Mycobacterium, Staphylococcus, Streptococcus, Pseudomonas, Klebsiella, Haemophilus, or Mycoplasma. In another aspect, the pathogen is a fungus selected from: Aspergillus flavus, Aspergillus fumigatus, Aspergillus nidulans, Aspergillus niger, Aspergillus terreus, Aspergillus ustus, Candida albicans, Candida alibicans, Candida glabrata, Candida lipolytica, Candida tropicalis, Candida tropicalis, Cryptococcus neoformans, Cryptococcus neoformas, Fusarium moniliforme, Geotricum candidum, Microsporum canis, Mucor circillelloides, Penicillium aurantiogriseum, Penicillium expansum, Penicillium italicum, Penicillium marneffei, Penicllium marneffeii, Rhizopus oryzaee, Sporotlirix schenckii, Syncephalastrum racemosum, Trichophyton mentagrophytes, Trichophyton rubrum, and a combination thereof. In another aspect, the tumor cells are derived from a tumor biopsy resected from a patient or cultured tumor cells. In another aspect, the method further comprises staining the one or more cutaneous layers at the site of administration prior to the imaging step. In another aspect, the method further comprises determining a number and type of recruited immune cells and immune cell-induced structural changes in the one or more cutaneous layers at the site of administration, wherein the immune cells are impartial markers for determining a potency of a pathogen/tumor-specific cellular immune response without bias associated with nonfighter T cells.

### BRIEF DESCRIPTION OF THE DRAWINGS

For a more complete understanding of the features and advantages of the present invention, reference is now made to the detailed description of the invention along with the accompanying figures and in which:

FIGS. 1A to 1E compare the control groups (FIG. 1A, FIG. 1D, FIG. 1E) with immunized mice 3 days after shaving/brushing/topical application of the influenza virus A/PR/8/34 (PR8) (FIG. 1B and FIG. 1C).

FIG. 2 shows the formation of “stratum immunis” in a mouse following topical application of H₂O₂-inactivated coronavirus (CoV) MHV1 particles.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

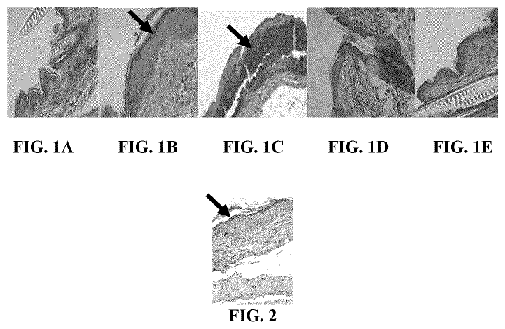

The present inventor developed a novel personalized cellular immunity assay (PCIA) by topical application of live or inactivated pathogen particles onto skin using a patch, which recruits tissue-resident T cells that target the pathogen epitopes along with other supporting cells to the patch site with bias associated with the MIA nonfighter T cell excluded. As shown in FIGS. 1A to 1E and FIG. 2, topical application of influenza virus (IFV) A/PR/8/34 (PR8) or coronavirus (CoV) MHV1 particles onto immunized mice rapidly recruited a swarm of immune cells to the patch site. On occasions, the infiltrated immune cells formed a new layer (tentatively dubbed “stratum immunis”) between stratum corneum and stratum granulosum.

Since changes in cutaneous architecture could be noninvasively visualized by imagers such as raster-scanning optoacoustic mesoscopy (RSOM) (Attia et al., 2019), the inventor developed the PCIA of the present invention by topical application of inactivated nonreplicating pathogen particles followed by noninvasive sectioning of the patch site with an imager. By way of explanation, but in no way a limitation of the present invention, it is hypothesized that the virus-recruited immune cells in the outer layer of skin (FIGS. 1A to 1E and FIG. 2) principally include tissue-resident immune cells, including Runx3-expressing T cells (Milner et al., 2017), without contamination by MIA nonfighter T cells that tend to enter the circulation. Critical immune cell populations usually reside within tissues and are not accessed by conventional blood draws used to monitor the immune response (Milner et al., 2017; Shin and Iwasaki, 2013). Thus, unlike binding assays commonly performed, the PCIA allows an impartial assessment of protective cellular immunity by excluding MIA T cells. Moreover, there is synergy between conventional antibody assays and PCIA to generate a more comprehensive analysis of protective immunity in a wide variety of vaccine/disease settings. It is demonstrated herein that a flood of immune cells are recruited to a patch site by topical application of viral particles (FIGS. 1A to 1E and FIG. 2). It further demonstrates that topical application of viral particles elicited a more measurable immune response surrounding viral particles along the interface than their counterparts injected intradermally (FIGS. 1A to 1E). Again, by way of explanation, but in no way a limitation of the present invention, it is hypothesized that because outer layer of skin is in constant contact with pathogens found in the environment the outer layer of skin is more immunocompetent than the inner cutaneous layers due to the necessity to deploy the most competent immune cells along the border to ward off constant infections.

Although topical application of viral particles recruited a flood of immune cells to the patch site in immunized animals (FIGS. 1A to 1E and FIG. 2), it is unlikely that immunity-driven structural changes will occur in the cutaneous architecture if immune cells should not be recruited to the patch site in sufficient numbers when there is no cellular immunity to the pathogen or when the cellular immune response is weak. Thus a noninvasive imager can detect a robust cellular immune response that remodels the cutaneous architecture whereas a weak cellular immune response has to be detected by immunohistochemical (IHC) analysis of a tiny subset of epidermis underneath the patch.

PCIA is an informative, non-invasive (or minimally invasive) assay that is the only assay that allows real-time detection of tissue-resident immune cells, which can be characterized by immunohistochemical (IHC) analysis following percutaneous fine-needle biopsy or surgical-excision biopsy (Catalano et al., 2020). Analysis of immunity-driven cutaneous changes by an imager, streamlines the assay process and make it more patient friendly by eliminating surgical procedures; however, imager analysis is not a prerequisite for PCIA to make medical contributions. Further, the high resolution of current/future cellular telephones, cameras, and smart watches should allow for the detection of the results at home or in a primary care setting. Further, the thickness or density changes can be measured using, e.g., ultrasound.

As shown in FIGS. 1A to 1E, no immune cells were recruited into the outer layer of skin in a non-immunized mouse 3 days after shaving/brushing (FIG. 1E); in a non-immunized mouse 3 days after shaving/brushing/topical application of PR8 (FIG. 1A); or in an immunized mouse 3 days after intradermal injection of PR8 (FIG. 1D). In contrast, a swarm of immune cells formed a new layer (tentatively dubbed “stratum immunis”; marked by an arrow) between stratum corneum and stratum granulosum in immunized mice 3 days after shaving/brushing/topical application of PR8 (FIG. 1B and FIG. 1C); the epidermis-dermis layers were also thickened in these immunized mice (FIG. 1B and FIG. 1C) compared to their counterparts in control groups (FIGS. 1A, 1D, and 1E).

Compelling evidence supports that topical application of an airborne virus can recruit immune cells to the outermost layer of skin to ward off invading viruses. The thickness of “stratum immunis” allows quantitative determination of the potency of an anti-virus cellular immune response without contamination by MIA T cells. In addition to the formation of “stratum immunis,” the topical application of PR8 particles also induced edema between dermis and muscle underneath the patch (FIG. 1C). The changes in cutaneous architecture induced by topical application of airborne viruses are amenable to imager analysis in support of the development of PCIA.

A weak cellular immune response that induces the formation of a thin layer of “stratum immunis” without inducing appreciable cutaneous landscape changes (FIG. 1B) may be missed by imager analysis. Notably, it is a common practice by dermatologists to resect a tiny piece of skin, which may be performed to detect weak cellular immunity by IHC analysis of recruited immune cells following percutaneous fine-needle biopsy or surgical-excision biopsy in clinical settings (Catalano et al., 2020). To eliminate the hazards associated with an infectious pathogen, as shown in FIG. 2, topical application of H₂O₂-inactivated CoV MHV1 particles could also induce the formation of “stratum immunis” underneath a patch in an A/J mouse.

Methods. Experimental cellular immunity assay for gauging cellular immunity against PR8 without contamination by MIA T cells following topical application of PR8 particles. IFV PR8 particles (2.6×10⁶ TCID₅₀ in 0.05 ml) were administered to the outer layer of abdominal skin by topical application onto control and immunized young (1-month old) female Balb/c mice using the 3M Tegaderm patch after ablation of stratum corneum with a toothbrush as described (Zhang et al., 2006). The skin underneath the patch was resected for histological analysis (H&E stain) 3 days after shaving/brushing, shaving/brushing/topical application of PR8, or intradermal injection of PR8. (FIG. 1A) A non-immunized mouse with skin shaved and brushed followed by topical application of PR8; (FIG. 1B) a mouse immunized by i.n. instillation of AdPR8.HA1 (1×10⁸ ifu in 0.05 ml) followed by shaving/brushing/topical application of PR8 37 days post-immunization; (FIG. 1C) a mouse immunized by i.n. instillation of PR8 (5.2×10³ TCID₅₀—a sublethal dose in 0.01 ml) on Day 0, immunized again by i.n. instillation of AdPR8.HA1 (1×10⁸ ifu in 0.05 ml) on Day 1, and immunized the third time by i.n. instillation of PR8 (1×10⁶ TCID₅₀ in 0.02 ml) on Day 9, followed by shaving/brushing/topical application of PR8 on Day 15; (FIG. 1D) a mouse immunized by i.n. instillation of AdPR8.HA1 (1×10⁸ ifu in 0.05 ml) on Day 0 followed by intradermal injection of PR8 (2.6×10⁶ TCID₅₀ in 0.05 ml) on Day 37; (FIG. 1E) A non-immunized mouse with skin shaved and brushed without PR8 administration; There were 2 (FIG. 1C and FIG. 1E) or 3 (FIGS. 1A, 1B, 1D) animals per group. Skin tissues underneath a patch were resected 3 days post-topical application, fixed, sectioned, stained with H&E, and were photographed under a 40× objective lens in a Keyence BZ-X800 series all-in-one fluorescence microscope.

FIG. 2 shows the formation of “stratum immunis” in an A/J mouse following topical application of H₂O₂-inactivated CoV MHV1 particles. A new layer of “stratum immunis” was formed underneath a patch 8 days after topical application of H₂O₂-inactivated CoV MHV1 particles in an immunized A/J mouse. It is conceivable that “stratum immunis” principally consists of immune cells including T cells that react to inactivated MHV1 particles in the outer layer of skin. H₂O₂-inactivated CoV MHV1 particles (6.5×10⁵ TCID₅₀ in 0.05 ml; titer determined by its PBS control) were administered to the outer layer of abdominal skin by topical application onto a female A/J mouse that survived challenge with a sublethal dose of MHV1 using the 3M Tegaderm patch after ablation of stratum corneum with a toothbrush. MHV1 was inactived by incubating live viruses in 2% H₂O₂ for 2.5 hours; and subsequently eliminated H₂O₂ by changing buffer to V20 using the VIVASPIN TURBO 100,000 MWCO; viruses incubated with PBS served as the control for determining the recovery rate of MHV1 particles. The skin underneath the patch was resected for histological analysis (H&E stain) 8 days post-patch application. Skin sections were photographed under a 40× objective lens in a Keyence BZ-X800 series all-in-one fluorescence microscope.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

### REFERENCES

Attia, A. B. E., Balasundaram, G., Moothanchery, M., Dinish, U. S., Bi, R., Ntziachristos, V., and Olivo, M. (2019). A review of clinical photoacoustic imaging: Current and future trends. Photoacoustics 16, 100144.
Catalano, O., Varelli, C., Sbordone, C., Corvino, A., De Rosa, D., Vallone, G., and Wortsman, X. (2020). A bump: what to do next? Ultrasound imaging of superficial soft-tissue palpable lesions. J Ultrasound 23, 287-300.
Davis, M. M. (2020). T cell analysis in vaccination. Curr Opin Immunol 65, 70-73.
Flaxman, A., and Ewer, K. J. (2018). Methods for measuring T-cell memory to vaccination: from mouse to man. Vaccines (Basel) 6, 43.
Gilbert, P. B., Montefiori, D. C., McDermott, A. B., Fong, Y., Benkeser, D., Deng, W., Zhou, H., Houchens, C. R., Martins, K., Jayashankar, L., et al. (2022). Immune correlates analysis of the mRNA-1273 COVID-19 vaccine efficacy clinical trial. Science 375, 43-50.
Milner, J. J., Toma, C., Yu, B., Zhang, K., Omilusik, K., Phan, A. T., Wang, D., Getzler, A. J., Nguyen, T., Crotty, S., et al. (2017). Runx3 programs CD8+ T cell residency in non-lymphoid tissues and tumours. Nature 552, 253-257.
Shin, H., and Iwasaki, A. (2013). Tissue-resident memory T cells. Immunol Rev 255, 165-181.
Takahashi, T., and Iwasaki, A. (2021). Sex differences in immune responses. Science 371, 347-348.
Theel, E. S., Slev, P., Wheeler, S., Couturier, M. R., Wong, S. J., and Kadkhoda, K. (2020). The role of antibody testing for SARS-CoV-2: Is there one? J Clin Microbiol 58, e00797-00720.
Wang, B., Wang, L., Kong, X., Geng, J., Xiao, D., Ma, C., Jiang, X., and Wang, P. (2020). Long-term coexistence of SARS-CoV-2 with antibody response in COVID-19 patients. J Med Virol 92, 1684-1689.
Wright, P. F., Hoen, A. G., Ilyushina, N. A., Brown, E. P., Ackerman, M. E., Wieland-Alter, W., Connor, R. I., Jegaskanda, S., Rosenberg-Hasson, Y., Haynes, B. C., et al. (2016). Correlates of Immunity to Influenza as Determined by Challenge of Children with Live, Attenuated Influenza Vaccine. Open Forum Infect Dis 3, ofw108.
Zhang, J., Shi, Z., Kong, F. K., Jex, E., Huang, Z., Watt, J. M., Van Kampen, K. R., and Tang, D. C. (2006). Topical application of Escherichia coli-vectored vaccine as a simple method for eliciting protective immunity. Infect Immun 74, 3607-3617.

What is claimed is:

1. A method of measuring a cellular immune response, the method comprising:
topically applying pathogen particles or tumor cells to a site on an outermost layer of skin, wherein the pathogen particles or tumor cells can recruit immune cells to a site of administration;
imaging changes in cutaneous architecture driven by infiltration of immune cells at the site after at least one day from the applying step; and
characterizing one or more structural changes to one or more cutaneous layers at the site of administration using an imager.

2. The method of claim 1, wherein the pathogen particles comprise pathogens inactivated by at least one of γ-irradiation, H₂O₂, or heat.

3. The method of claim 1, wherein the pathogen particles comprise pathogens selected from a virus, a bacterium, or a fungus.

4. The method of claim 3, wherein the virus is selected from: influenza virus, coronavirus, respiratory syncytial virus, rhinovirus, or measles virus.

5. The method of claim 3, wherein the bacterium is selected from: Bacillus, Clostridium, Mycobacterium, Staphylococcus, Streptococcus, Pseudomonas, Klebsiella, Haemophilus, or Mycoplasma.

6. The method of claim 3, wherein the fungus is selected from: Aspergillus flavus, Aspergillus fumigatus, Aspergillus nidulans, Aspergillus niger, Aspergillus terreus, Aspergillus ustus, Candida albicans, Candida alibicans, Candida glabrata, Candida lipolytica, Candida tropicalis, Candida tropicalis, Cryptococcus neoformans, Cryptococcus neoformas, Fusarium moniliforme, Geotricum candidum, Microsporum canis, Mucor circillelloides, Penicillium aurantiogriseum, Penicillium expansum, Penicillium italicum, Penicillium marneffei, Penicllium marneffeii, Rhizopus oryzaee, Sporotlirix schenckii, Syncephalastrum racemosum, Trichophyton mentagrophytes, Trichophyton rubrum, and a combination thereof.

7. The method of claim 1, wherein the tumor cells are derived from a tumor biopsy resected from a patient or cultured tumor cells.

8. The method of claim 1, further comprising staining the one or more cutaneous layers at the site of administration prior to the imaging step.

9. The method of claim 1, further comprising determining a number and type of recruited immune cells and immune cell-induced structural changes in one or more cutaneous layers at the site of administration, wherein the immune cells are impartial markers for determining a potency of a pathogen/tumor-specific cellular immune response without bias associated with nonfighter T cells.

10. The method of claim 1, further comprising detecting a protective cellular immune response against a wide variety of pathogens without contamination by nonfighter T cells, wherein an assay is selected from at least one of a quantitative, a noninvasive, or a personalized assay.

11. A composition for detecting a protective cellular immune response comprising: live or inactivated virus particles, bacterium cells, fungus cells, or tumor cells that recruit immune cells to a site of administration, wherein inactivation is by heat, γ-irradiation, or H₂O₂ formulated for topical administration at a site of administration for detection of the protective cellular immune response.

12. The composition of claim 11, wherein the composition further comprises an abrasive.

13. A method of measuring a cellular immune response, the method comprising:
topically applying pathogen particles or tumor cells to a site of administration on an outermost layer of skin, wherein the pathogen particles or tumor cells can recruit immune cells to the site of administration;
imaging changes in cutaneous architecture driven by infiltration of immune cells at the site after at least one day from the applying step;
characterizing one or more structural changes to one or more cutaneous layers at the site administration using an imager; and
detecting a protective cellular immune response against the pathogen particles or tumor cells, wherein an assay is selected from at least one of a quantitative, a noninvasive, or a personalized assay.

14. The method of claim 13, wherein the pathogen particles comprise pathogens inactivated by at least one of γ-irradiation, H₂O₂, or heat.

15. The method of claim 13, wherein the pathogen is a virus, a bacterium, a fungus, or a tumor cell.

16. The method of claim 15, wherein the pathogen is a virus selected from: influenza virus, coronavirus, respiratory syncytial virus, rhinovirus, or measles virus.

17. The method of claim 15, wherein the pathogen is a bacterium selected from: Bacillus, Clostridium, Mycobacterium, Staphylococcus, Streptococcus, Pseudomonas, Klebsiella, Haemophilus, or Mycoplasma.

18. The method of claim 15, wherein the pathogen is a fungus selected from: Aspergillus flavus, Aspergillus fumigatus, Aspergillus nidulans, Aspergillus niger, Aspergillus terreus, Aspergillus ustus, Candida albicans, Candida alibicans, Candida glabrata, Candida lipolytica, Candida tropicalis, Candida tropicalis, Cryptococcus neoformans, Cryptococcus neoformas, Fusarium moniliforme, Geotricum candidum, Microsporum canis, Mucor circillelloides, Penicillium aurantiogriseum, Penicillium expansum, Penicillium italicum, Penicillium marneffei, Penicllium marneffeii, Rhizopus oryzaee, Sporotlirix schenckii, Syncephalastrum racemosum, Trichophyton mentagrophytes, Trichophyton rubrum, and a combination thereof.

19. The method of claim 15, wherein the tumor cells are derived from a tumor biopsy resected from a patient or cultured tumor cells.

20. The method of claim 13, further comprising at least one of:
staining the one or more cutaneous layers at the site of administration prior to the imaging step; or
determining a number and type of recruited immune cells and immune cell-induced structural changes in the one or more cutaneous layers at the site of administration, wherein the immune cells are impartial markers for determining a potency of a pathogen/tumor-specific cellular immune response without bias associated with nonfighter T cells.
